(19) United States
(12) Patent Application Publication
(10) Pub. No.: US 2017/0281728 A1
(43) Pub. Date: Oct. 5, 2017

(54) METHOD FOR TREATING CANCER USING TETRADRINE

(71) Applicant: Hari K. KOUL, Shreveport, LA (US)

(72) Inventor: Hari K. KOUL, Shreveport, LA (US)

(73) Assignee: Board of Supervisors of Louisiana State University and Agricultural and Mechanical College, Baton Rouge, LA (US)

(21) Appl. No.: 15/479,759

(22) Filed: Apr. 5, 2017

Related U.S. Application Data
(60) Provisional application No. 62/318,473, filed on Apr. 5, 2016.

(51) Int. Cl. A61K 38/19; A61K 31/4741

(52) U.S. Cl. CPC A61K 38/191; A61K 31/4741

(57) ABSTRACT

A method of treating a cancer disease or pre-disease condition in a mammal comprising administering a first pharmaceutical composition including a therapeutically effective amount of a first therapeutic, wherein the first therapeutic is tetrandrine, a tetrandrine derivative, or pharmacologically acceptable salts, solvates, esters, amides, clathrates, stereoisomers, enantiomers, prodrugs or analogs thereof, or a combination thereof.

### CROSS REFERENCE TO RELATED APPLICATIONS/PRIORITY

The present invention claims priority to U.S. Provisional Patent Application No. 62/318,473 filed Apr. 5, 2016, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

The subject matter disclosed herein was partly supported by the National Cancer Institute of the National Institutes of Health under Award Number NCI RO1-CA161880.

### BACKGROUND OF THE INVENTION

Tumor necrosis factor-related apoptosis-inducing-ligand (TRAIL) and tumor necrosis factor-alpha (TNF-α, TNF, tumor necrosis factor, cachexin, or cachectin) each induce apoptosis in the majority of cancer cells. TRAIL is a protein functioning as a ligand that induces the process of cell death called apoptosis. TRAIL is a cytokine that is produced and secreted by most normal tissue cells. It causes apoptosis primarily in tumor cells, by binding to certain death receptors. TNF-α is a cell signaling protein (cytokine) involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. TRAIL and its receptors have been used as the targets of several anti-cancer therapeutics since the mid-1990s, however these therapeutics have not shown significant survival benefit. The inventor observed that resistance to TRAIL and TNF-α occurs in many tumors including Prostate Cancer. For the foregoing reasons, there is a pressing, but seemingly irresolvable need for sensitizing cancer cells to treatment with TRAIL and TNF-α.

### SUMMARY OF THE INVENTION

Wherefore, it is an object of the present invention to overcome the above mentioned shortcomings and drawbacks associated with the current technology. The present invention is directed to methods and apparatuses that satisfy the above shortcomings and drawbacks. The method and apparatus include a method of treating a cancer disease or pre-disease condition in a mammal comprising administering a first pharmaceutical composition including a therapeutically effective amount of a first therapeutic, wherein the first therapeutic is tetrandrine, a tetrandrine derivative, or pharmacologically acceptable salts, solvates, esters, amides, clathrates, stereoisomers, enantiomers, prodrugs or analogs thereof, or a combination thereof. According to a further embodiment, the method further comprises administering a second pharmaceutical composition including a therapeutically effective amount of a second therapeutic wherein the second therapeutic is one of TNF-related apoptosis-inducing ligand (TRAIL), tumor necrosis factor-alpha (TNF-α), and pharmacologically acceptable salts, solvates, esters, amides, clathrates, stereoisomers, enantiomers, prodrugs or analogs thereof, or a combination thereof. According to a further embodiment, the method comprises administering a second pharmaceutical composition including a therapeutically effective amount of a second therapeutic wherein the second therapeutic is one of TNF-related apoptosis-inducing ligand (TRAIL), tumor necrosis factor-alpha (TNF-α), and pharmacologically acceptable salts, solvates, esters, amides, clathrates, stereoisomers, enantiomers, prodrugs or analogs thereof, or a combination thereof. According to a further embodiment, the first pharmaceutical composition is administered before the second pharmaceutical composition. According to a further embodiment, the first pharmaceutical composition is administered one of 10 minutes, 30 minutes, 1 hour, 2 hours, 5 hours, 10 hours, 15 hours, 18 hours, 24 hours, 36 hours, 48 hours, and 72 hours before the second pharmaceutical composition. According to a further embodiment, the first therapeutic is administered in one of 5 times, 10 times, 50 times, 100 times, 500 times, 1,000 times, 2,000 times, and 5,000 times the amount, measured in moles, of the second therapeutic. According to a further embodiment, the second therapeutic is one of TRAIL, a pharmacologically acceptable salt, solvate, ester, amide, clathrate, stereoisomer, enantiomer, prodrug or analog thereof, and a combination thereof. According to a further embodiment, the second therapeutic is one of TNF-α, a pharmacologically acceptable salt, solvate, ester, amide, clathrate, stereoisomer, enantiomer, prodrug or analog thereof, and a combination thereof. According to a further embodiment, the second therapeutic includes both TRAIL and TNF-α, or pharmacologically acceptable salts, solvates, esters, amides, clathrates, stereoisomers, enantiomers, prodrugs or analogs thereof, or a combination thereof. According to a further embodiment, the mammal is human. According to a further embodiment, the first and the second pharmaceutical composition is administered via one of topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, and oral administration.

The disclosed invention further relates to products and methods of treating including a therapeutic product comprising a first pharmaceutically active agent being one of tetrandrine, a tetrandrine derivative, or pharmacologically acceptable salts, solvates, esters, amides, clathrates, stereoisomers, enantiomers, prodrugs or analogs thereof, or a combination thereof and a second pharmaceutically active agent being one of TNF-related apoptosis-inducing ligand (TRAIL), tumor necrosis factor-alpha (TNF-α), and pharmacologically acceptable salts, solvates, esters, amides, clathrates, stereoisomers, enantiomers, prodrugs or analogs thereof, or a combination thereof. According to a further embodiment, the second pharmaceutically active agent is one of TRAIL, pharmacologically acceptable salts, solvates, esters, amides, clathrates, stereoisomers, enantiomers, prodrugs or analogs thereof, and a combination thereof. According to a further embodiment, the second pharmaceutically active agent is one of TNF-α, pharmacologically acceptable salts, solvates, esters, amides, clathrates, stereoisomers, enantiomers, prodrugs or analogs thereof, and a combination thereof. According to a further embodiment, the second pharmaceutically active agent is one of both TRAIL and TNF-α, and pharmacologically acceptable salts, solvates, esters, amides, clathrates, stereoisomers, enantiomers, prodrugs or analogs thereof, or a combination thereof.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a set of nine pairs of x-ray film exposures, one top and one bottom, and a densitometry graph displaying the relative pixel density of the different film exposures from a top film exposure compared to a bottom film exposure;

FIG. 2A-2C are three Western Blots of and DR4, DR5 and TNFR1 levels of C4-2b cells subjected to various treatment conditions;

FIG. 3 is a densitometry graph of DR4 fold increase as shown on western blots for LNCaP cells subjected to various treatment conditions;

FIG. 4 is a densitometry graph of DR5 fold increase as shown on western blots for LNCaP cells subjected to various treatment conditions;

FIG. 5 is a densitometry graph of TNFR1 fold increase as shown on western blots for LNCaP cells subjected to various treatment conditions;

FIG. 6 is a graph of relative mRNA expression of DR4 normalized to GAPDH;

FIG. 7 is a graph of relative mRNA expression of DR5 normalized to GAPDH;

FIG. 8 is a graph of relative mRNA expression of TNFR1 normalized to GAPDH;

FIG. 9 is a graph of relative value of the ratio of DR4/GAPDH;

FIG. 10 is a graph of relative value of the ratio of DR5/GAPDH;

FIG. 11 is a graph of relative value of the ratio of TNFR1/GAPDH;

FIG. 12 is three photographs of crystal violet staining of LNCap cells subjected to variable treatment conditions;

FIG. 13 is a graph of MTT assay survival rate of LNCap cells subjected to the same variable treatment conditions of the LNCap cells of FIG. 12;

FIG. 14 is three photographs of crystal violet staining of C4-2b cells subjected to variable treatment conditions; and

FIG. 15 is a graph of MTT assay survival rate of C4-2b cells subjected to the same variable treatment conditions of the C4-2b cells of FIG. 14.

### DETAILED DESCRIPTION OF THE PREFERRED EMBODIMENTS

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

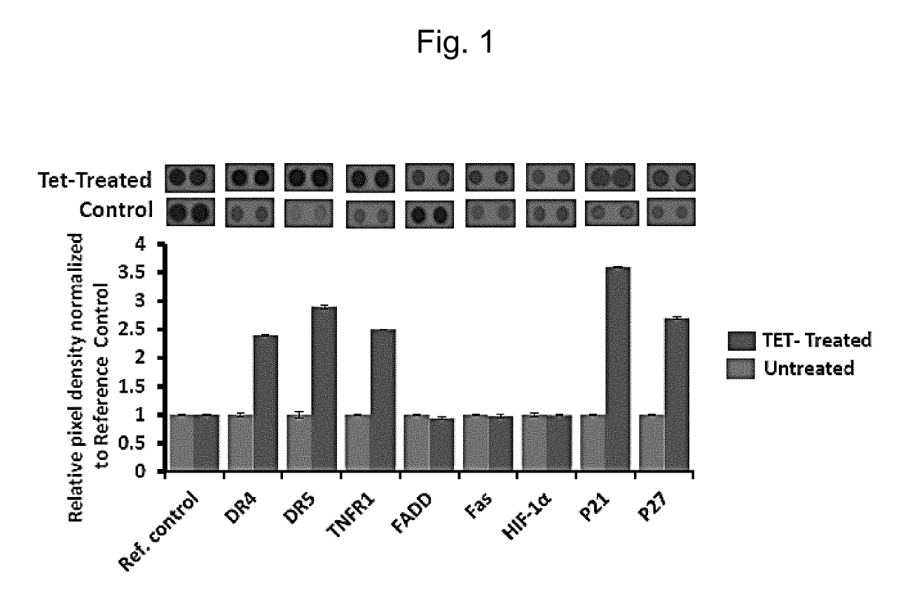

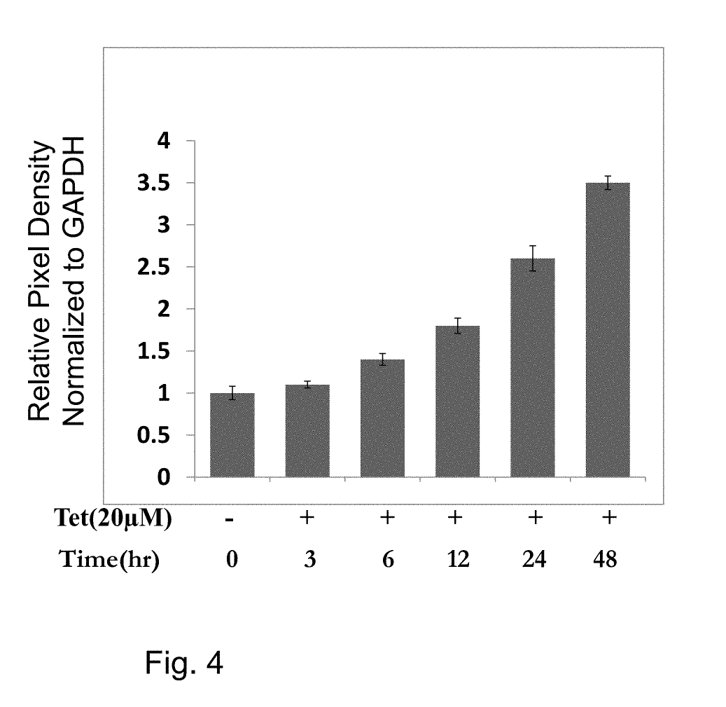

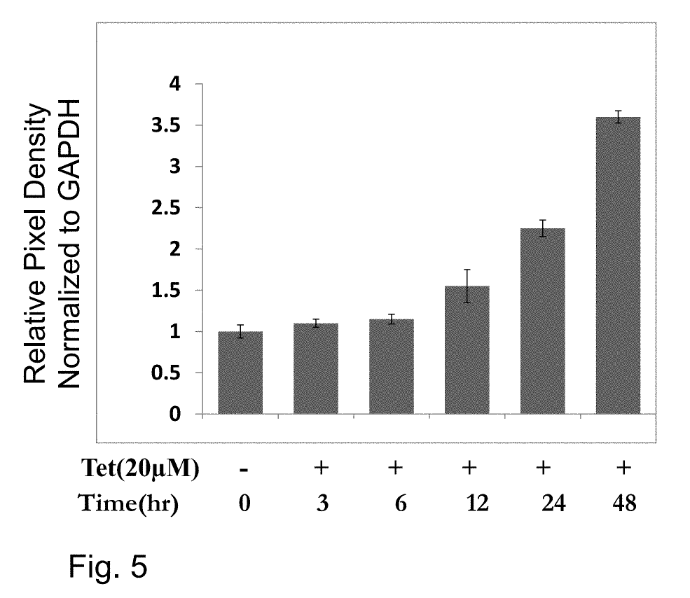

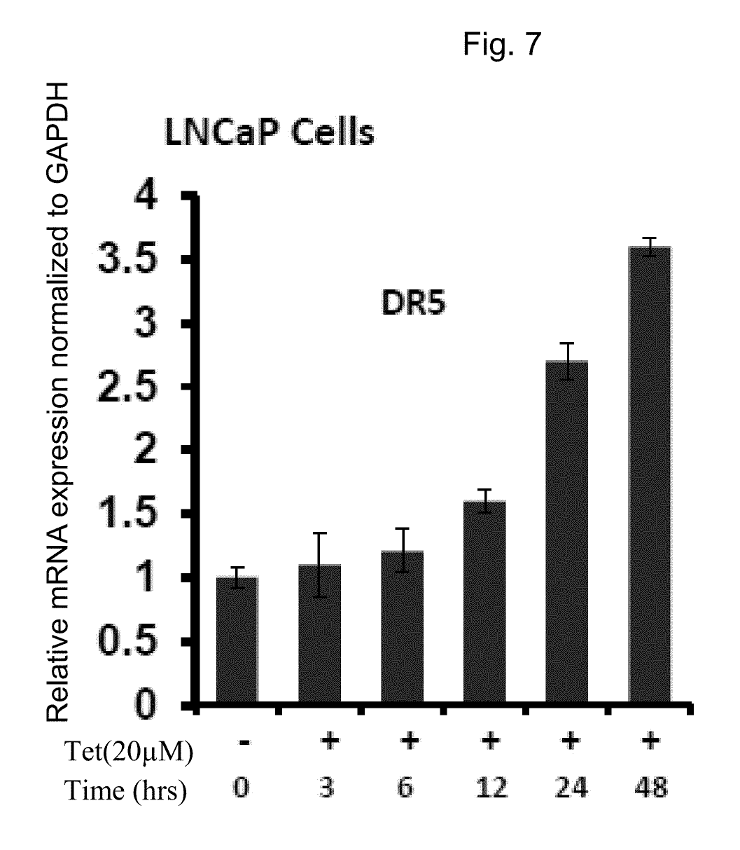

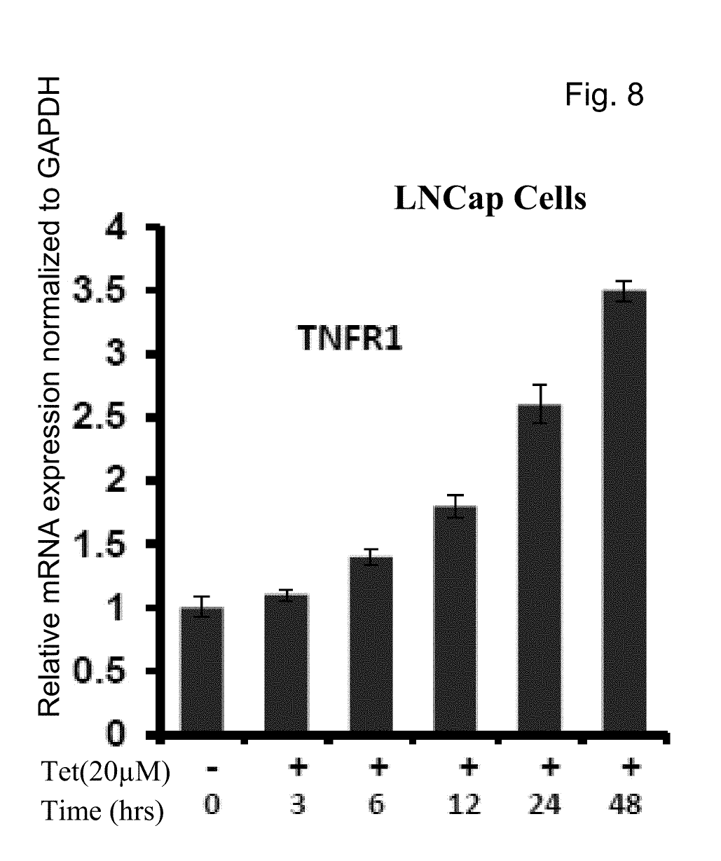

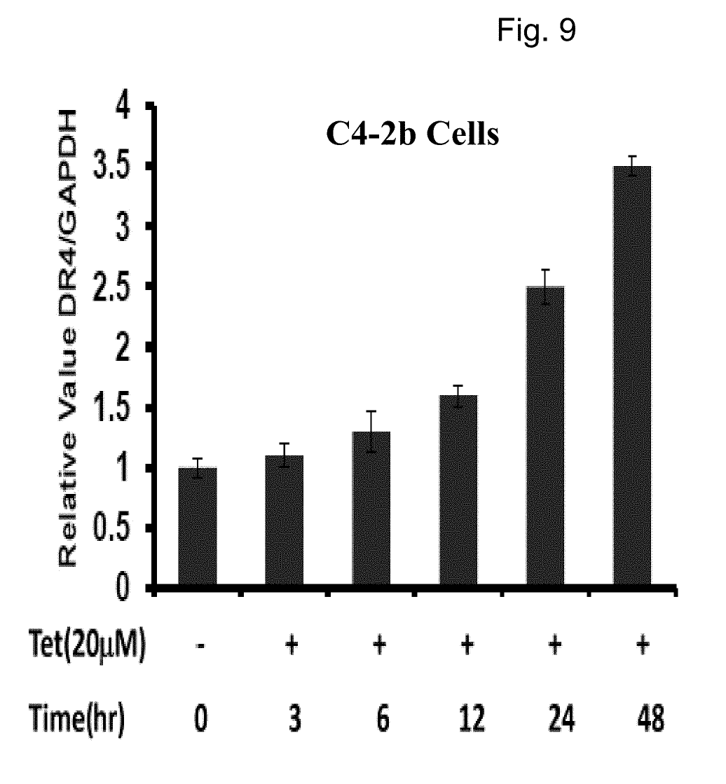

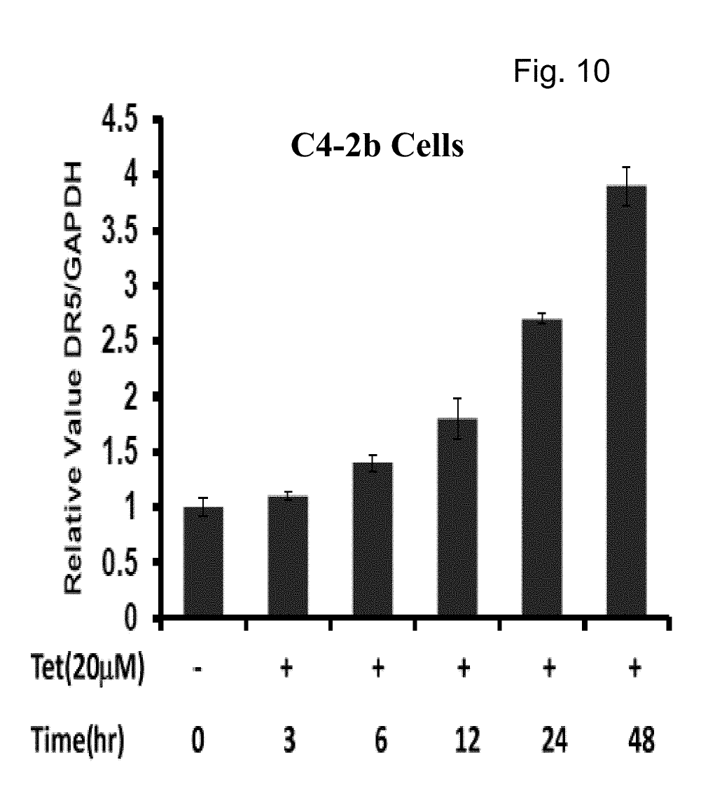

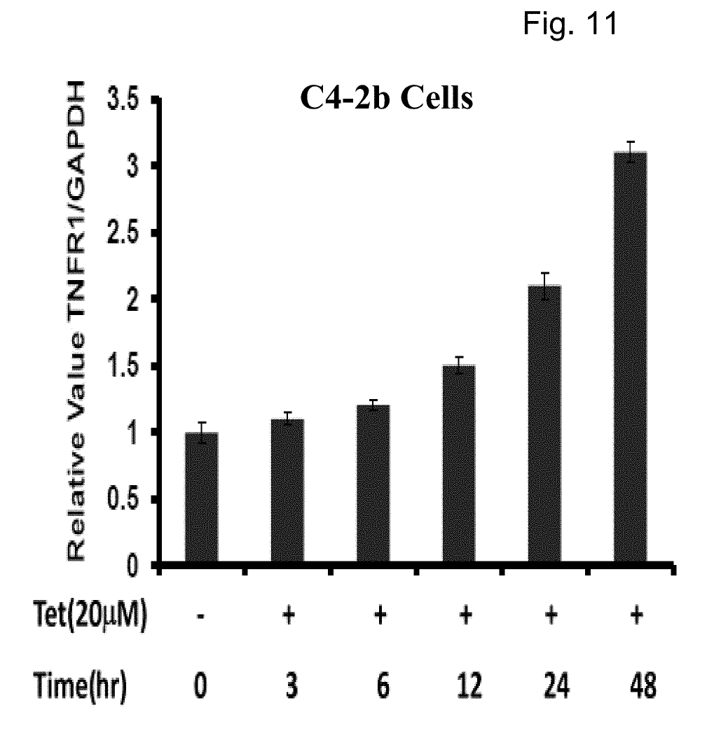

Turning now to FIGS. 1-15, a brief description concerning the various components of the present invention will now be briefly discussed. TNF-related apoptosis-inducing ligand (TRAIL/Apo2L) has been used to preferentially kill cancer cells without destroying the majority of healthy normal cells. TRAIL has also been designated CD253 (cluster of differentiation 253) and TNFSF10 (tumor necrosis factor (ligand) super family, member 1. TRAIL is preferentially cytotoxic to cancer cells over normal cells. Interestingly, the TRAIL signaling pathway is blocked in normal cells due to a high expression of decoy receptors and FLICE-like inhibitory protein, which inhibits caspase 8, caspase 10, and apoptosis. Though TRAIL is a promising anti cancer agent, the effectiveness of TRAIL is affected by drug resistance, resulting in poor survival outcomes of patients with cancer. This is one of the major impediments to use of TRAIL as a single agent against several tumor types. New agents that improve efficacy of TRAIL are highly warranted.

Resistance to apoptosis is one of the mechanisms used by cancer cells to overcome toxicity of therapeutic agents. Cancer cells have evolved numerous strategies to resist cell death induction via the extrinsic pathway. Extrinsic apoptosis pathway involves death receptors to induce apoptosis. TRAIL induces apoptosis mainly through DR-mediated pathways. TRAIL binds on the death receptors, death receptor 4 (DR4) and death receptor 5 (DR5) and activates apoptotic pathway selectively in cancer cells. DR4, also known as TRAIL receptor 1 (TRAILR1) and tumor necrosis factor receptor super family member 10A (TNFRSF10A), is a cell surface receptor of the TNF-receptor super family that binds TRAIL and mediates apoptosis. The protein encoded by this gene is a member of the TNF-receptor super family. This receptor is activated by tumor necrosis factor-related apoptosis inducing ligand (TNFSF10/TRAIL/APO-2L), and thus transduces cell death signal and induces cell apoptosis. DR5, also known as TRAIL receptor 2 (TRAILR2) and tumor necrosis factor receptor super family member 10B (TNFRSF10B), is a cell surface receptor of the TNF-receptor super family that binds TRAIL and mediates apoptosis. The protein encoded by this gene is a member of the TNF-receptor super family, and contains an intracellular death domain. This receptor can be activated by tumor necrosis factor-related apoptosis inducing ligand (TNFSF10/TRAIL/APO-2L), and transduces apoptosis signal.

Tumor necrosis factor receptor 1 (TNFR1), also known as tumor necrosis factor receptor super family member 1A (TNFRSF1A) and CD120a, is a ubiquitous membrane receptor that binds TNF-α. The protein encoded by this gene is a member of the tumor necrosis factor receptor super family, which also contains TNFRSF1B. This protein is one of the major receptors for the TNF-α. This receptor can activate the transcription factor NF-κB, mediate apoptosis, and function as a regulator of inflammation.

Specifically targeting the extrinsic pathway to trigger apoptosis in tumor cells is attractive for cancer therapy since death receptors have a direct link to the cell's death machinery. The inventor investigated Tetrandrine (TET, a bis-benzylisoquinoline alkaloid originally isolated from the roots of Stephania tetrandra) as a TRAIL and TNF-α sensitizer anti cancer agent, which is attractive as it has a low toxicity and less resistance in normal cells. The inventor discloses methods to induce DR4, DR5, and TNFR1 in cancer cells by use of TET. The inventor also discloses that exposure of TRAIL or TNF-60 resistant cancer cells to sub-lethal doses of Tetrandrine results in TRAIL or TNF-α sensitization and restoration of TRAIL or TNF-α induced apoptosis. The results of the disclosed experiments evidence that sub-lethal doses of TET would be achievable in a patient to sensitize cancer cells in vivo without being lethal to the patient.

Tetrandrine (C₃₈H₄₂N₂O₆, TET, Tet) is a bis-benzylisoquinoline alkaloid and is a calcium channel blocker. Tetrandrine may be biosynthesized from a free radical coupled dimerization of S—N-methylcoclaurine. A chemical structure for TET is shown below:

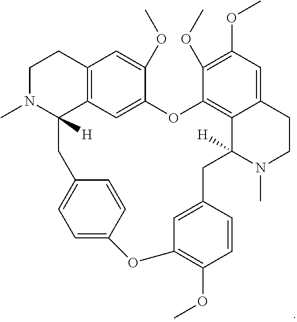 [SMILES: COc1ccc2cc1Oc1ccc(cc1)C[C@H]1c3cc(c(OC)cc3CCN1C)Oc1c(OC)c(OC)cc3c1[C@H](C2)N(C)CC3]

Derivatives of TET are also contemplated as being part of the present disclosure.

LNCaP and C4-2b cells were used as model cell lines that show resistance to TRAIL. The inventors have experimentally demonstrated that TET promotes apoptosis in prostate cancer cells in vitro and in vivo. In the present study we evaluated the effects of TET alone or in combination with TRAIL and TNF-α. Prostate cancer cell lines LNCaP and LNCaP derived C4-2b were used for this study. Cell lines were treated with TET, TRAIL, or TNF-αalone or in various combinations for different time points. mRNA expression was quantitated via Real Time-PCR and changes in protein expression were analyzed by Western Blot analysis. Cytotoxicity was evaluated using the Crystal violet and MTT survival assay.

Turning to FIG. 1, LNCaP and C4-2b cells did not show a significant response following exposure to TRAIL or TNF-α. LNCaP cells were either untreated or treated with 20 μM TET for 24 hours. Arrays were incubated with 400 μg of each cell lysate. Data shown in FIG. 1 are from a 1 minute exposure to X-ray film. Densitometry analysis shows fold increase for DR4, DR5 and TNFR1. Fold increase was calculated with the density of untreated LNCaP cells as a reference. Averages are representative of 2 independent experiments. Error bars represent standard deviation.

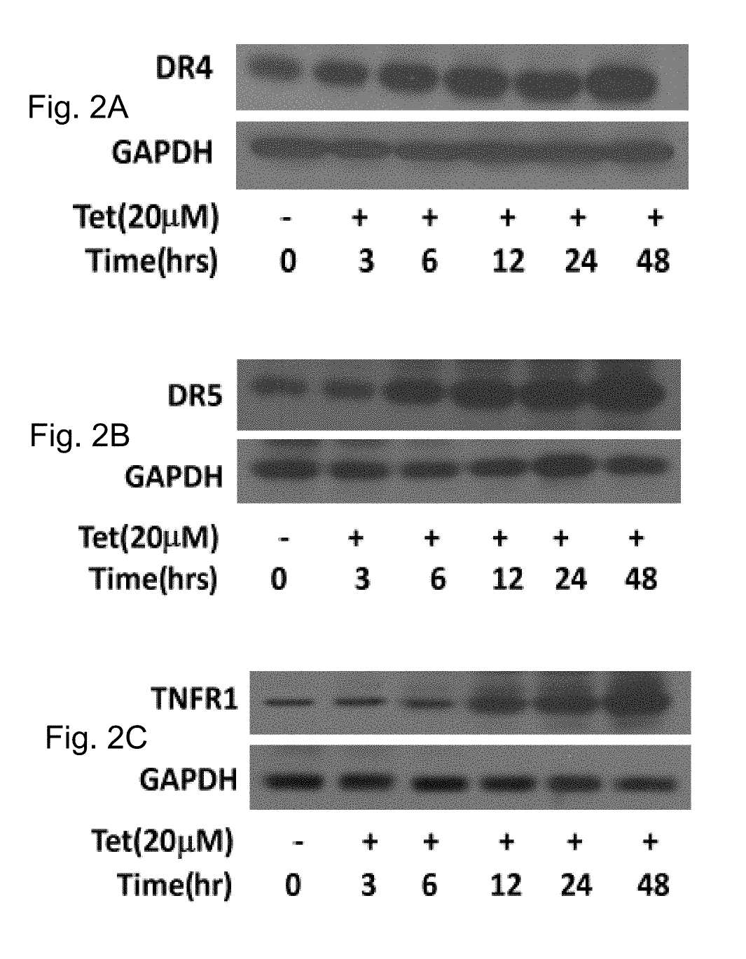

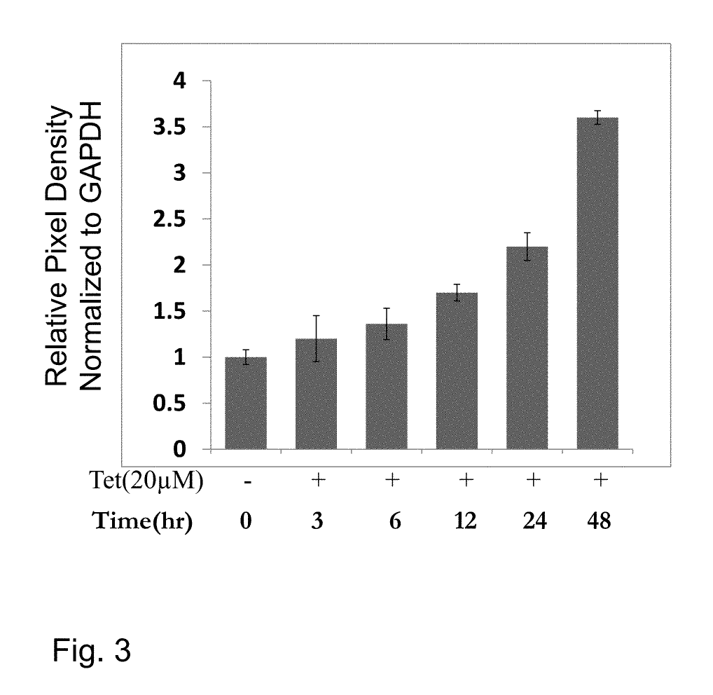

Turning to FIGS. 2A-2C, C4-2b cells were treated with 20 μM TET for 0-48 hours. After the treatments cells were lysed and DR4, DR5 and TNFR1 levels were detected by western blotting. Similarly, in FIGS. 3-5, densiomentry fold increase for DR4, DR5 and TNFR1 western blots is shown. The fold increase was calculated with the density of untreated LNCaP cells as a reference. GAPDH was used as loading control. Averages are representative of 3 independent experiments. Error bars represent standard deviation.

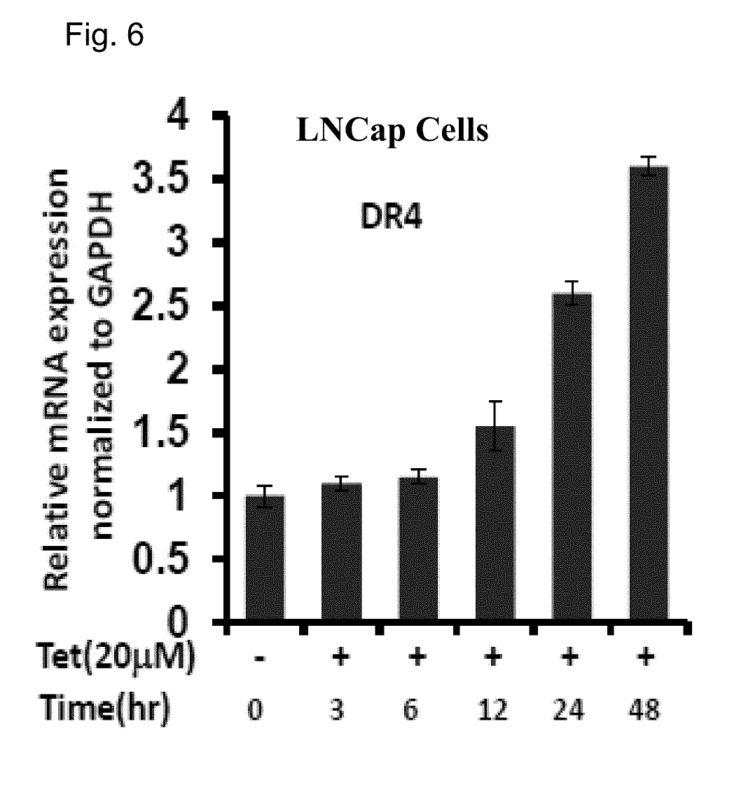

Turning to FIGS. 6-11, LNCaP and C4-2b cells were incubated in the presence of 20 μM of Tet for different time points (0-48 hours). Total RNA was prepared from the cells and cDNA was transcribed. Reverse transcriptase PCR was performed to determine the mRNA levels of DR4, DR5 and TNF-α. GAPDH was used for normalization. Each data point represents mean+/−standard deviation of three independent gels. Exposure of LNCaP and C4-2b cells to TET resulted in increased mRNA and protein levels of death receptors DR4, DR5 and TNFR1.

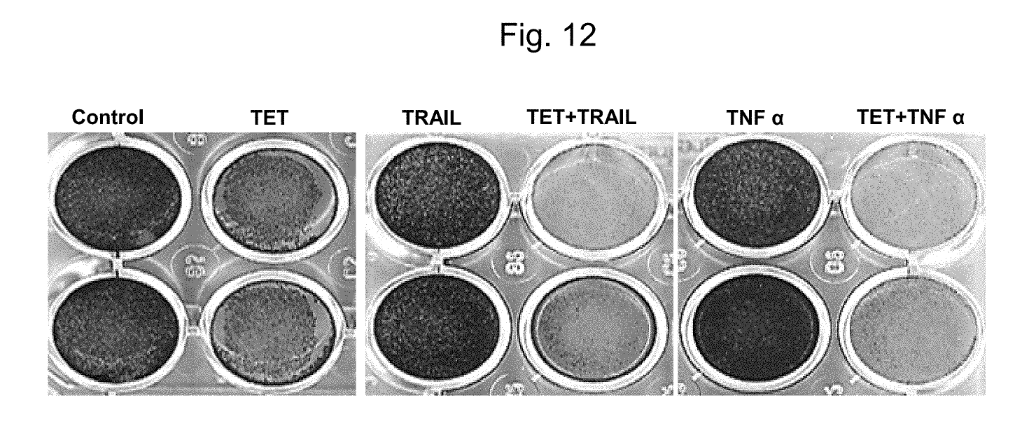

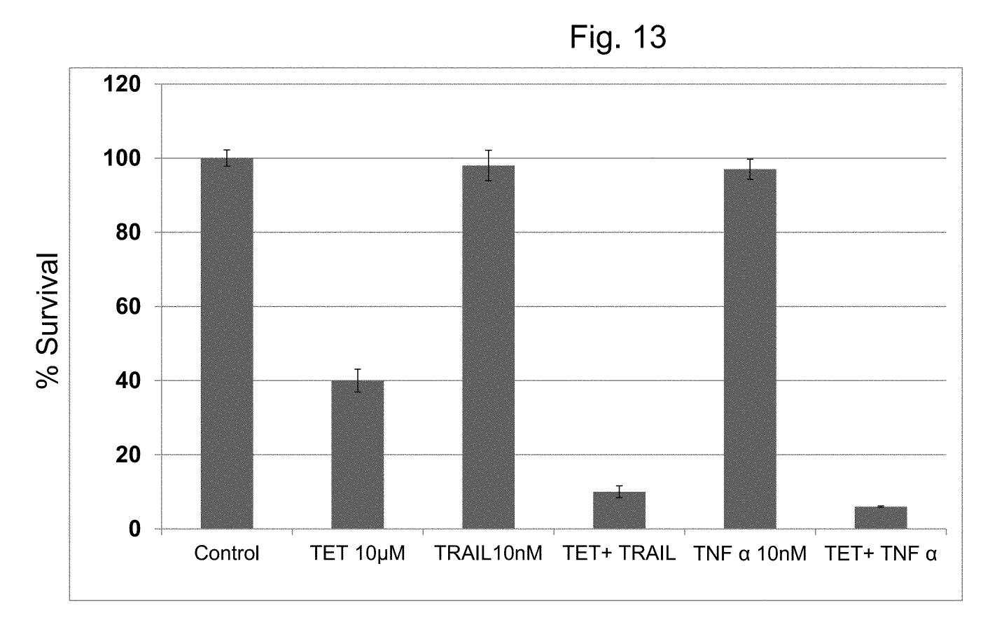

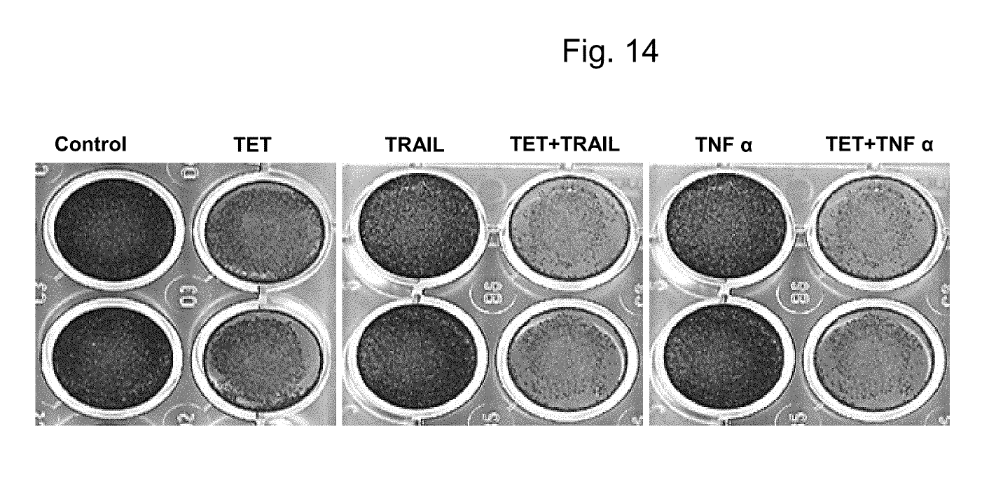

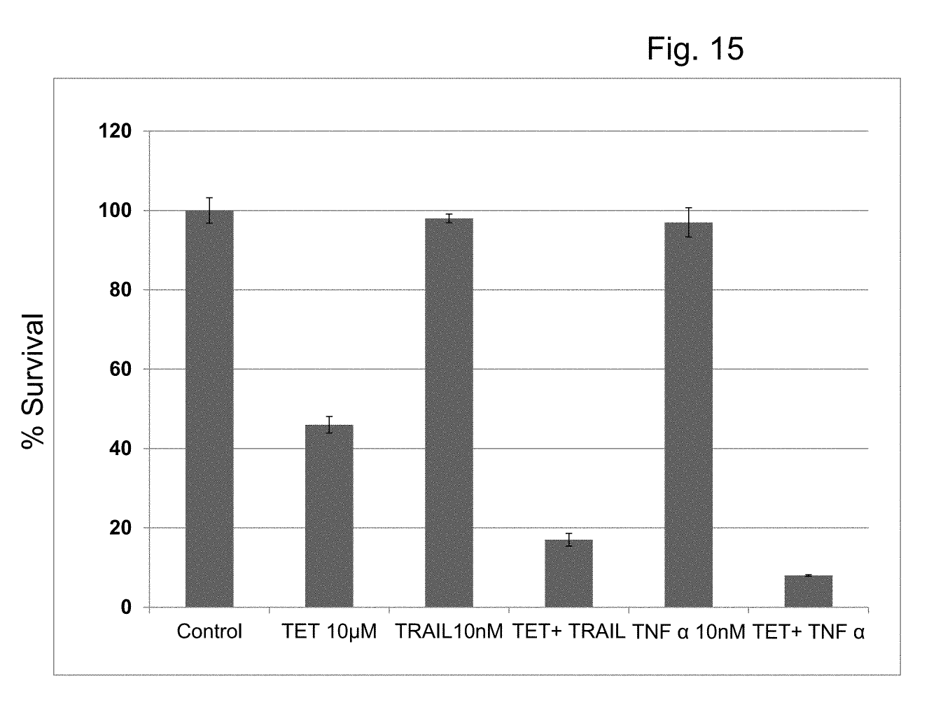

Turning to FIGS. 12-15, pretreatment of both cell lines with TET for 12 hours, followed by TRAIL and TNF-α treatment for another 24 hours increased the apoptosis inducing potential of TRAIL and TNF-α in these cells. Exposure of TET treated cells in vitro to soluble h-TRAIL and TNF-α in combination with TET resulted in synergistic apoptosis response. For FIGS. 12 and 14, LNCap (FIG. 12) and C4-2b (FIG. 14) cells treated with or without TET, TRAIL, and TNFα were crystal violet stained. Wells are representative of at least three repeats. For FIGS. 13 and 15, LNCap (FIG. 13) and C4-2b (FIG. 15) cells were subjected to MTT assay using the same experimental plan as in FIGS. 12, 14 respectively. Data were normalized to the untreated control at the respective concentrations of TET, TRAIL and TNF-α. Data are representative of at least three repeats. These results show that TET by inducing expression of DR4, DR5 and TNFR1 sensitizes cancer cells to TRAIL and TNF-α. Exposure of TET treated cells in vitro to soluble TRAIL and TNF-α in combination with TET resulted in synergistic apoptosis response. These results show that TET by inducing expression of DR4, DR5 and TNFR1 sensitizes cancer cells to TRAIL and TNF-α.

The results provide evidence for a novel therapeutic strategy in which the sequential administration of TET followed by TRAIL and/or TNF-α and/or contemporaneous administration of TET with TRAIL and/or TNF-α would be used as an effective strategy for eradicating solid tumors in general and prostate cancer in particular.

As used herein, the term “tetrandrine” or “TAT” includes tetrandrine, tetrandrine derivatives, or any pharmacologically acceptable salt, solvate, ester, amide, clathrate, stereoisomer, enantiomer, prodrug, or analog thereof, or a combination thereof.

As used herein, the term “active agent” includes TAT, TRAIL, and TNF-α, alone and in combination. The term active agent may also be referred to as the active compound, active ingredient, active material, the inventive compound and/or the active drug substance.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active results from the use of an enteric coating of an oral medication (e.g., an oral dosage form).

The term an “effective amount” of an agent, as used herein, includes that amount sufficient to effect beneficial or desired results, such as clinical results, and, as such, an “effective amount” may depend upon the context in which it is being applied.

The terms “extended release” or “sustained release” interchangeably includes a drug formulation that provides for gradual release of a drug over an extended period of time, e.g., 6-12 hours or more, compared to an immediate release formulation of the same drug. Preferably, although not necessarily, extended release results in substantially constant blood levels of a drug over an extended time period that are within therapeutic levels and fall within a peak plasma concentration range that is between, for example, 0.05-20 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coaterie®), hdroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release,” as used herein, includes that the agent (e.g., TAT or any pharmacologically acceptable salt, solvate, ester, amide, clathrate, stereoisomer, enantiomer, prodrug, or analog thereof, or a combination thereof), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing an active agent described herein (e.g., TAT or any pharmacologically acceptable salt, solvate, ester, amide, clathrate, stereoisomer, enantiomer, prodrug, or analog thereof, or a combination thereof), formulated with a pharmaceutically acceptable excipient, and typically manufactured or sold with the approval of a governmental regulatory agency as part of a therapeutic regimen for the treatment of disease in a mammal, especially with the mammal being a human.. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes an ingredient other than the active agents described herein (for example, a vehicle capable of suspending or dissolving the active compound) and having the properties of being nontoxic and non-inflammatory in a patient. Excipients may include, for example: anti adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palm itate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylam monium, methylam ine, dimethylam ine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., cancer), or may refer to a treatment of a pre-disease state. Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of TAT, or any pharmacologically acceptable salt, solvate, ester, amide, clathrate, stereoisomer, enantiomer, prodrug, or analog thereof, or a combination thereof.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also refer to delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

The present compounds can be prepared from readily available starting materials using the methods and procedures known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one of ordinary skill in the art by routine optimization procedures.

Pharmaceutical Compositions: The methods described herein can also include the administrations of pharmaceutically acceptable compositions that include TAT or any pharmacologically acceptable salt, solvate, ester, amide, clathrate, stereoisomer, enantiomer, prodrug, or analog thereof, or a combination thereof. Pharmaceutical compositions and dosage forms of the invention comprise one or more active ingredients in relative amounts and formulated so that a given pharmaceutical composition or dosage form causes TRAIL or TNF-α sensitization in cancer cells and restoration of TRAIL or TNF-α induced apoptosis of cancer cells, or any pharmacologically acceptable salt, solvate, ester, amide, clathrate, stereoisomer, enantiomer, prodrug, or analog thereof, or a combination thereof, optionally in combination with one or more additional active agents. When employed as pharmaceuticals, any of the present active agents can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention (e.g., TAT, TRAIL, and/or TNF-α or any pharmacologically acceptable salts, solvates, esters, amides, clathrates, stereoisomers, enantiomers, prodrugs, or analogs thereof, or a combination thereof) can be administered alone, combined, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

The pharmaceutical compositions can be formulated so as to provide immediate, extended, or delayed release of the active ingredient after administration to the patient by employing procedures known in the art.

In addition to other dosages listed herein, the pharmaceutical compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the active agent may be mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration. The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active agent by controlling the dissolution and/or the diffusion of the active agent substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the administered therapeutic or drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxym ethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings: The pharmaceutical compositions formulated for oral delivery, such as tablets or capsules of the present invention can be coated or otherwise compounded to provide a dosage form affording the advantage of delayed or extended release. The coating may be adapted to release the active agent in a predetermined pattern (e.g., in order to achieve a controlled release formulation) or it may be adapted not to release the active agent until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the active agent is released in the lower gastrointestinal tract.

In addition to coatings that effect delayed or extended release, the solid tablet compositions may include a coating adapted to protect the composition from unwanted chemical changes (e.g., chemical degradation prior to the release of the active agent). The coating may be applied on the solid dosage form in a similar manner as that described in Encyclopedia of Pharmaceutical Technology, vols. 5 and 6, Eds. Swarbrick and Boyland, 2000.

Parenteral Administration: Within the scope of the present invention are also parenteral depot systems from biodegradable polymers. These systems are injected or implanted into the muscle or subcutaneous tissue and release the incorporated active agent over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery: Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992)

Oral transmucosal absorption is generally rapid because of the rich vascular supply to the mucosa and allows for a rapid rise in blood concentrations of the therapeutic or active agent (“American Academy of Pediatrics: Alternative Routes of Drug Administration—Advantages and Disadvantages (Subject Review),” Pediatrics, 100(1):143-152, 1997).

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, menthol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acidand chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art.

Formulations for sublingual administration can also be used, including powders and aerosol formulations. Exemplary formulations include rapidly disintegrating tablets and liquid-filled soft gelatin capsules.

Dosing Regimens: The present methods for treating cancer disease or pre-disease state are carried out by administering one or more TAT or any pharmacologically acceptable salt, solvate, ester, amide, clathrate, stereoisomer, enantiomer, prodrug, or analog thereof, or a combination thereof for a time and in an amount sufficient to result in stabilization and/or reversal of cancer disease or pre-disease state symptoms. The amount and frequency of administration of the compositions can vary depending on, for example, what is being administered, the state of the patient, and the manner of administration. The dosage is likely to depend on such variables as the type and extent of progression of the cancer disease or pre-disease state, the severity of the cancer disease or pre-disease state, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of cancer disease or pre-disease state or slowing its progression.

The amount of active agent per dose can vary. For example, a subject can receive from about 0.1 μg/kg to about 50,000 μg/kg. Generally, the active agent is administered in an amount such that the peak plasma concentration ranges from 1.50 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1.0, or 2.0 mg/kg. In another embodiment, the administered dosage can range from 0.05-5 mmol of an active agent (e.g., 0.089-3.9 mmol) or 0.1-50 μmol of an active agent (e.g., 0.1-25 μmol or 0.4-20 μmol).

The frequency of treatment may also vary. The subject can be treated one or more times per day with the active agent (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

KITS: Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce incidence, duration, and or severity of cancer disease or pre-disease state.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of treating a cancer disease or pre-disease condition in a mammal comprising:
administering a first pharmaceutical composition including a therapeutically effective amount of a first therapeutic;
wherein the first therapeutic is tetrandrine, a tetrandrine derivative, or pharmacologically acceptable salts, solvates, esters, amides, clathrates, stereoisomers, enantiomers, prodrugs or analogs thereof, or a combination thereof.

2. The method of claim 1 further comprising administering a second pharmaceutical composition including a therapeutically effective amount of a second therapeutic wherein the second therapeutic is one of TNF-related apoptosis-inducing ligand (TRAIL), tumor necrosis factor-alpha (TNF-α), and pharmacologically acceptable salts, solvates, esters, amides, clathrates, stereoisomers, enantiomers, prodrugs or analogs thereof, or a combination thereof.

3. The method of claim 2 wherein the first pharmaceutical composition is administered before the second pharmaceutical composition.

4. The method of claim 2 wherein the first pharmaceutical composition is administered one of 10 minutes, 30 minutes, 1 hour, 2 hours, 5 hours, 10 hours, 15 hours, 18 hours, 24 hours, 36 hours, 48 hours, and 72 hours before the second pharmaceutical composition.

5. The method of claim 2 wherein the first therapeutic is administered in one of 5 times, 10 times, 50 times, 100 times, 500 times, 1,000 times, 2,000 times, and 5,000 times the amount, measured in moles, of the second therapeutic.

6. The method of claim 2 wherein the second therapeutic is one of TRAIL, a pharmacologically acceptable salt, solvate, ester, amide, clathrate, stereoisomer, enantiomer, prodrug or analog thereof, and a combination thereof.

7. The method of claim 2 wherein the second therapeutic is one of TNF-α, a pharmacologically acceptable salt, solvate, ester, amide, clathrate, stereoisomer, enantiomer, prodrug or analog thereof, and a combination thereof.

8. The method of claim 2 wherein the second therapeutic includes both TRAIL and TNF-α, or pharmacologically acceptable salts, solvates, esters, amides, clathrates, stereoisomers, enantiomers, prodrugs or analogs thereof, or a combination thereof.

9. The method of claim 2 wherein the mammal is human.

10. The method of claim 2 wherein the first and the second pharmaceutical composition is administered via one of topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, and oral administration.

11. A therapeutic product comprising:
a first pharmaceutically active agent being one of tetrandrine, a tetrandrine derivative, pharmacologically acceptable salts, solvates, esters, amides, clathrates, stereoisomers, enantiomers, prodrugs or analogs thereof, and a combination thereof; and
a second pharmaceutically active agent being one of TNF-related apoptosis-inducing ligand (TRAIL), tumor necrosis factor-alpha (TNF-α), and pharmacologically acceptable salts, solvates, esters, amides, clathrates, stereoisomers, enantiomers, prodrugs or analogs thereof, and a combination thereof.

12. The therapeutic product of claim 11 wherein the second pharmaceutically active agent is one of TRAIL, pharmacologically acceptable salts, solvates, esters, amides, clathrates, stereoisomers, enantiomers, prodrugs or analogs thereof, and a combination thereof.

13. The therapeutic product of claim 11 wherein the second pharmaceutically active agent is one of TNF-α, pharmacologically acceptable salts, solvates, esters, amides, clathrates, stereoisomers, enantiomers, prodrugs or analogs thereof, and a combination thereof.

14. The therapeutic product of claim 11 wherein the second pharmaceutically active agent is one of both TRAIL and TNF-α, and pharmacologically acceptable salts, solvates, esters, amides, clathrates, stereoisomers, enantiomers, prodrugs or analogs thereof, or a combination thereof.
